(19) United States
(12) Patent Application Publication
(10) Pub. No.: US 2020/0297889 A1
(43) Pub. Date: Sep. 24, 2020

(54) AIRBORNE MICROORGANISMS NEUTRALIZING SYSTEM AND METHOD OF NEUTRALIZING AIRBONE MICROORGANISM

(71) Applicant: ALFA LAVAL CORPORATE AB, Lund (SE)

(72) Inventors: Patrick M. Leach, Lund (SE); Michael A. Harvey, Lund (SE); Jonathan Bell, Lund (SE)

(73) Assignee: ALFA LAVAL CORPORATE AB, Lund (SE)

(21) Appl. No.: 16/361,826

(22) Filed: Mar. 22, 2019

(51) Int. Cl. A61L 9/20; B05B 15/00; B05B 9/04; B01D 53/14; B01D 53/00; B01D 53/26; B01D 53/28

(52) U.S. Cl. CPC A61L 9/20; B05B 15/00; B05B 9/0403; B01D 53/1425; B01D 53/007; B01D 2259/804; B01D 53/28; A61L 2209/12; B01D 2252/103; B01D 2252/60; B01D 53/263

(57) ABSTRACT

A system for neutralizing airborne microorganisms includes a conditioning section for accommodating a flow of air, the conditioning section including an air inlet; an air outlet; a packing chamber located downstream relative to the air inlet, a distribution chamber located downstream relative to the packing chamber and located above the packing chamber, the distribution chamber including at least one spray nozzle for spraying a liquid desiccant into the distribution chamber towards the packing chamber; a sump located below the packing chamber; a pump for propelling the liquid desiccant from the sump to the at least one spray nozzle; a droplet collection chamber located downstream relative to the at least one spray nozzle for removing droplets from the flow of air flowing through the droplet collection chamber; and a UV disinfection chamber located downstream relative to the droplet collection chamber and upstream relative to the air outlet.

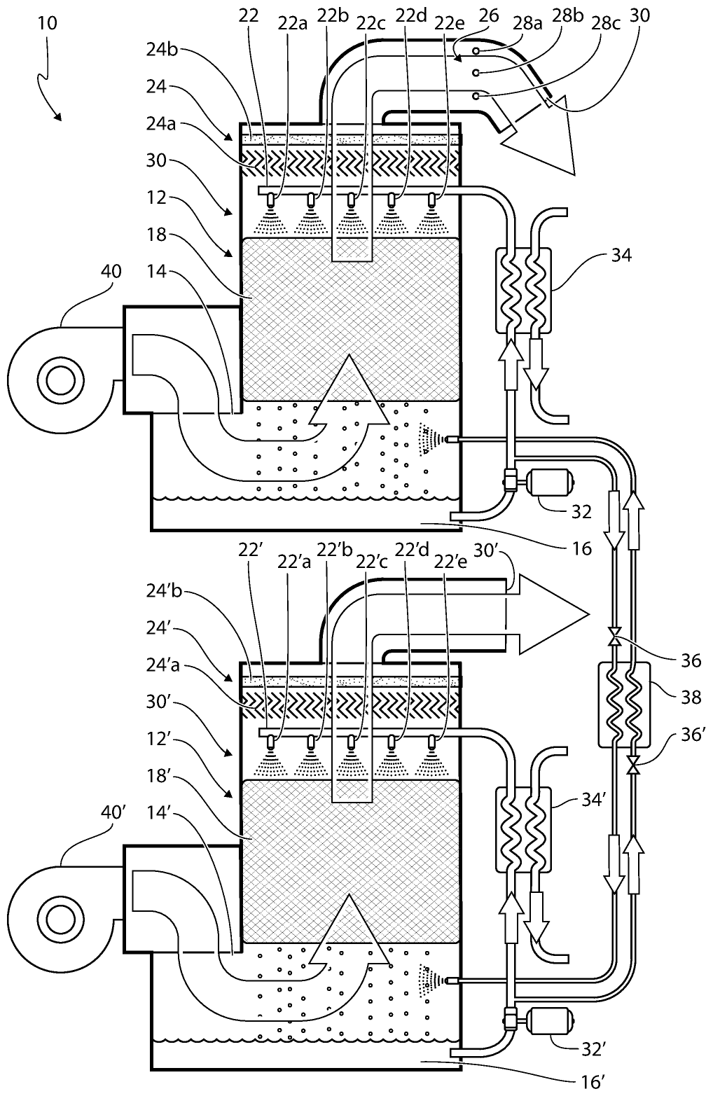

### BACKGROUND OF THE INVENTION

### 1. Field of the Invention

The present invention relates to a system and a method for neutralizing airborne microorganisms in indoor environments, especially within food and beverage processes and healthcare settings.

### 2. Description of Related Art

Control and eradication of pathogenic microorganisms within indoor environments is an increasing concern, within food and beverage production and within healthcare facilities. By using appropriate HVAC (Heating, Ventilation and Air Conditioning) the number of microorganisms in the indoor environment may be reduced. This will aid in protecting food and beverage processes from viable airborne contaminants and improve patient outcome in healthcare settings.

It is known to use UVGI (Ultra Violet Germicidal Irradiation) for the purpose of inactivating or killing microorganisms, see https://en.wikipedia.org/wiki/Ultraviolet_germicidal_irradiation. Using UVGI is not as effective on moving air, as the exposure times are reduced compared to air at rest. The use of UVGI irradiation will typically produce a 1-4.5 LOG reduction of the microorganisms in the air.

There are various background art devices for cleaning indoor air. For example, U.S. Pat. No. 6,843,835 B2 relates to an indoor air cleaning apparatus using a scrubbing liquid for the removal of impurities dissolved or dispersed in the ambient air by contacting the scrubbing liquid. The scrubbing liquid comprises a non-evaporative liquid that can remove at least some volatile organic compounds and gaseous pollutants from ambient air containing such pollutants. Microorganism may be destroyed by the additional use of antimicrobial agents. The scrubbing liquid may be irradiated by UV light to catalyse the destruction of organic and biological contaminants by peroxides of photobleaching agents contained in the scrubbing liquid.

WO 2004/106812 A1 relates to a heat recovering ventilation apparatus for an air conditioning room. The incoming air is first irradiated by germicidal UV light and then exposed to a lithium chloride solution to absorb moisture from the air.

CN 2152170U relates to a full heat exchange air cleaner. During operation, the outdoor fresh air is first filtered through a dust filter to filter out dust impurities. The air then enters a heat exchanger which releases air of suitable temperature and humidity. The air is then sterilized by an ultraviolet germicidal lamp. The heat exchanger is made of a metal material having good thermal conductivity. A superabsorbent material is sandwiched between heat conducting sheets. The superabsorbent material is a cloth, porous nylon or foamed plastic sheet which is subjected to a water absorbing chemical lithium chloride for soaking treatment.

A known desiccant dehumidification system uses a desiccant medium which provides a lower vapor pressure than the surrounding air. The system is presented at http://www.kathabar.com/ and http://www.iisgroupllc. com/wp-content/uploads/2013/04/Kathabar-Technical-Bulletin.pdf. The system may be used for controlling temperature and humidity of indoor air, however, it may also act as an air scrubber to neutralize airborne microorganisms for bacteria-free air. The Kathabar system will typically produce a 1 LOG reduction of the microorganisms in the air.

In very sensitive environments, such as within food and beverage processes and healthcare settings, it is necessary to have near 100% elimination of viable microbial matter. It is thus an object according to the present invention to provide a system and a method for neutralizing airborne microorganisms in indoor environments, especially within food and beverage processes and healthcare settings.

### SUMMARY OF THE INVENTION

In a first aspect of the present invention, the above object is achieved by a system for neutralizing airborne microorganisms including a conditioning section for accommodating a flow of air, the conditioning section including an air inlet for receiving microorganism laden air and an air outlet for delivering microorganism free air, the conditioning section further comprising:

a packing chamber located downstream relative to the air inlet, the packing chamber being filled with a fibrous packing material,

a distribution chamber located downstream relative to the packing chamber and located above the packing chamber when the system is in its normal position of use, the distribution chamber including at least one spray nozzle for spraying a liquid desiccant into the distribution chamber towards the packing section,

a sump located below the packing chamber when the system is in its normal position of use for receiving the liquid desiccant which has penetrated the packing chamber from the distribution chamber through gravity,

a pump for propelling the liquid desiccant from the sump to the spray nozzle,

a droplet collection chamber located downstream relative to the spray nozzle for removing droplets from the flow of air flowing through the droplet collection chamber, and

a UV disinfection chamber located downstream relative to the droplet collection chamber and upstream relative to the air outlet, the UV disinfection chamber comprising a UV energy source for exposing the flow of air to UV irradiation.

The above system incorporates liquid desiccant technology. Most significantly the system continuously and systemically removes and renders harmless viable microbial matter as it passes through the conditioning section. This is accomplished via exposure to harmful osmotic stress, changes in the ionic charge environment and/or toxicity due to the high concentrations of metals. The resulting air will be free of microbial contaminants.

It is understood that the airborne microorganisms are contained in aerosols, i.e. droplets, in the flow of air. Generally, the system is able to inactivate both bacteria and spores. Specifically, the system can inactivate the following pathogens that are associated with hospital acquired infections and/or food compromising: Pseudomonas aeruginosa, Klebsiella, Acinetobacter, Staphylococcus aureus, Clostridium difficile, E. coli, Listeria and Salmonella.

The present system according to the first aspect includes a conditioning section used for removing microorganisms in ventilations systems for buildings, primarily for ventilation systems used in the food, beverage and healthcare industries. The air outlet is arranged to supply the interior of the building with dehumidified or humidified clean air.

The air inlet is typically connected to the outside of the building; however, the system is capable of accommodating both outside and return air and can incorporate a complete air pre-filtration section. The packing material is a fibrous material capable of receiving the liquid desiccant sprayed from the nozzle. As air passes through the packing chamber, microorganisms laden in the air stream flow into the fibrous packing material where they come in direct contact with the liquid desiccant. The fibrous packing material will be soaked with liquid desiccant and allows for a large contact surface between the microorganism containing aerosol droplets and the liquid desiccant. The liquid desiccant and the microorganisms bound to the liquid desiccant will precipitate into the sump by gravity. In the sump, the non-viable particulate matter is removed by a side-stream filter and the liquid desiccant is propelled back to the spray nozzle.

Upon passing through the packing material, the air continues to the distribution chamber which is flooded with aerosolized liquid desiccant from the spray nozzle. The spray nozzle forms part of a distribution header in the distribution chamber. The air is thereby further scrubbed by contact between the microorganism laden droplets in the air and the aerosolized liquid desiccant. The spray nozzle is directed towards the packing material and the liquid desiccant is thus sprayed towards the direction of the air stream.

The air then passes through the droplet collection chamber in which any aerosolized liquid in the air stream, primarily droplets of liquid desiccant, are separated from the flow of dry air which continues to the UV disinfection chamber. The liquids are collected and form larger droplets which flow downwards and eventually reach to the sump. The liquid in the sump is recirculated to the distribution header and propelled to the nozzles using a pump. The pump and the nozzles should be dimensioned such that an aerosolized liquid is generated by the nozzle.

Remaining viable microorganisms in the air after the liquid desiccant scrubbing will be deactivated by the UV irradiation causing permanent deactivation.

The result of the air flowing through the four different chambers, i.e. the packing chamber, the distribution chamber, the droplet collection chamber and the UV disinfection chamber, is a minimum deactivation of 99.999% of the viable microorganisms in the air, a rate substantially exceeding the effectiveness of each of the chambers alone or any previous similar device or method. The present invention can produce an average LOG reduction of at least 5.45+/−1.15 of microorganisms in the air, which is much higher than the UVGI and Kathabar systems alone. In particular, depending on the microorganisms, the present invention has been tested to produce results even higher that the above LOG reduction. For instance, for Staph, the present invention has obtained a log reduction of 5.75+/−0.24. Also, E. coli's average LOG reduction was 3.93+/−0.08, Listeria's LOG reduction was 4.09+/−0.08, and Enterobacter's average LOG reduction was 6.59+/−0.33. Finally, Salmonella showed a greater than 6.90 Net LOG reduction, Pseudomonas showed a greater than 4.73 Net LOG reduction and Klebsiella species showed a minimum net LOG reduction of 6.14. Overall, as mentioned above, these test results showed an average LOG reduction of at least 5.45+/−1.15.

According to a further embodiment of the first aspect, the liquid desiccant comprises a mixture of water and a salt, the salt being one of CaCl₂ or LiCl and the concentration of the salt in the mixture being between 10% and 60%, preferably between 20% and 45%. The exact concentration depends on the desired humidity of the air released from the system. A higher concentration of salt in the liquid desiccant solution will yield a less humid air as more humidity is absorbed by the liquid desiccant. A control system may be used for keeping temperature and humidity within desired levels.

According to a further embodiment of the first aspect, the UV energy source is an LED or a lamp such as a low-pressure UV lamp, preferably a low-pressure mercury lamp.

According to a further embodiment of the first aspect, the intensity of UVC energy inside the UV disinfection chamber is at least 10 W/m², preferably at least 50 W/m², more preferably at least 124 W/m². The UV disinfection device must have adequate UV energy to be able to irradiate the flow of air to achieve deactivation of viable microorganisms.

According to a further embodiment of the first aspect, the conditioner is made utilizing non-metallic construction with internal parts also non-metallic engineered plastics. In this way, corrosion may be avoided.

According to a further embodiment of the first aspect, the droplet collection chamber comprises a replaceable pad demister.

According to a further embodiment of the first aspect, the system further comprises a blower for generating a flow of air from the air inlet to the air outlet. A blower may be considered an integral part of a ventilation system.

According to a further embodiment of the first aspect, the flow of air defines a velocity less than 3 m/s, such as 2.235 m/s. The above velocities are suitable for achieving a proper disinfection of the flow of air.

According to a further embodiment of the first aspect, the UV disinfection chamber comprise an array or UV energy sources. In this way, it is easier to sufficiently irradiate the complete flow of air. The exposure to UV irradiation between the droplet collection chamber and the air outlet in combination with desiccant solution contact shall determine the effectiveness of the deactivation of microorganisms.

According to a further embodiment of the first aspect, the system further comprises a regenerating section for regenerating the water absorbing properties of the liquid desiccant.

According to a further embodiment of the first aspect, the regenerating section extends between a secondary air inlet for dry air and a secondary air outlet for humid air and defines a secondary flow direction, the regenerating section comprising:

a secondary packing chamber located downstream relative to the air inlet, the secondary packing chamber being filled with a secondary fibrous high efficiency packing material,

a secondary spray chamber located downstream relative to the secondary packing chamber and located above the secondary packing chamber when the system is in its normal position of use, the secondary spray chamber including at least one secondary spray nozzle for spraying the liquid desiccant into the spray chamber towards the secondary packing section,

a secondary sump located below the secondary packing chamber when the system is in its normal position of use for receiving the liquid desiccant from the secondary spray chamber which have penetrated the secondary packing chamber through gravity,

a secondary pump for pumping the liquid desiccant from the secondary sump to the secondary spray nozzle, and

a secondary heat exchanger between the secondary sump and the secondary spray nozzle for heating the liquid desiccant.

The regenerating section looks and works similar to the conditioning section; however, the liquid desiccant is heated before being sprayed toward the secondary packing section by the secondary nozzle instead of being cooled as in the conditioning section. By using outside air at the secondary air inlet, the liquid desiccant will release water to a passing stream of outside air. The liquid desiccant is then cooled to reestablish the water absorbing properties before being led back to the conditioning section.

In a second aspect of the present invention, the above object is achieved by a method of neutralizing airborne microorganisms comprising providing a conditioning section extending between an air inlet and an air outlet, the conditioning section further comprising:

a packing chamber located downstream relative to the air inlet, the packing chamber being filled with a fibrous packing material.

a distribution chamber located downstream relative to the packing chamber and located above the packing chamber when the system is in its normal position of use, the distribution chamber including at least one spray nozzle,

a sump located below the packing chamber when the system is in its normal position of use,

a pump,

a droplet collection chamber located downstream relative to the spray nozzle, and

an UV disinfection chamber located downstream relative to the droplet collection chamber and upstream relative to the air outlet, (the UV disinfection chamber comprising adequate UV energy for microbial inactivation),

the method comprising the steps of:

introducing microorganism laden air into the air inlet,

spraying a liquid desiccant into the distribution chamber towards the packing section by using the spray nozzle,

receiving the liquid desiccant which has penetrated the packing chamber from the distribution chamber through gravity into a sump,

propelling the liquid desiccant from the sump to the spray nozzle

removing droplets from the air flowing through the droplet collection chamber,

exposing the air within the UV disinfection chamber with adequate UV irradiation and,

receiving substantially microorganism free air at the air outlet.

The method according to the second aspect is preferably used together with the system according to the first aspect. The above method incorporates liquid desiccant technology. Most significantly, the method continuously and systemically removes and renders harmless viable microbial matter as it passes through the conditioning section. This is accomplished via exposure to harmful osmotic stress, changes in the ionic charge environment and/or toxicity due to the high concentrations of metals and UV irradiation. The resulting air will be free of microbial contaminants.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWING

The present invention will become more fully understood from the detailed description given hereinbelow and the accompanying drawings which are given by way of illustration only, and thus are not limitative of the present invention, and wherein:

The FIGURE shows a system for neutralizing airborne micororganims including a conditioning section and a regenerating section.

### DETAILED DESCRIPTION OF THE DRAWING

The FIGURE shows a system 10 for neutralizing airborne micororganims. The system comprises separate conditioning and regenerating sections 12, 12′. The conditioning section 12 comprises an air inlet 14 for receiving air from the outside. A blower 40 can be provided for generating a flow of air from the air inlet to the air outlet. The blower 40 may also be an integral part of the ventilation system in which the system 10 for neutralizing airborne microorganisms is installed. The outside air and/or return air may include harmful microorganisms contained in aerosols which may be introduced into the indoor air. In sensitive environments, such as the food/beverage industry and healthcare facilities/industry, these microorganisms may cause disease. Microorganims may include: Pseudomonas aeruginosa, Klebsiella, Acinetobacter, Staphylococcus aureus, Clostridium difficile, E. coli, Listeria and Salmonella.

The conditioning section 12 incorporates industrial-grade fiberglass construction with internal parts of non-metallic engineered plastics. The flow of air from the air inlet 14 is drawn through a sump area 16 with a 180° turn as shown by the arrow. The sump area 16 comprising a solution of liquid desiccant. The liquid desiccant within the system is preferably an aqueous solution of LiCl or CaCl₂ and can be adjusted to various concentration levels (20% to 45%) to accommodate the required temperature and humidity level for the corresponding conditioned space. The flow then continues into a high efficiency contact fibrous packing chamber 18 impregnated with liquid desiccant that allows for maximum surface contact of air to liquid desiccant.

Downstream relative to the packing chamber 18 and above the packing chamber 18 when the conditioning section is in its normal position of use, a distribution chamber 20 is located. The distribution chamber 20 includes a liquid desiccant spray distribution header 22. The spray distribution header includes one or more spray nozzles 22a-e for spraying solution into the distribution chamber 20 towards the packing chamber 18 and generating an aerosol of liquid desiccant in the distribution chamber 20. The spray nozzles 22a-e are capable of flooding the distribution chamber 20 with liquid desiccant at a specific particle size and distribution. The distribution header 22 is equipped with flow regulation.

Downstream relative to the distribution chamber 20 and above the distribution chamber 20 when the conditioning section is in its normal position of use, a droplet collection chamber 24 is located. The droplet collection chamber 24 comprises an engineered mist eliminator located directly downstream of the distribution chamber 20. In the droplet collection chamber 24, any droplets in the flow of air are removed. The droplet collection chamber 24 in the present example includes a Z-bar 24a and a demister pad 24b. Alternatively, the Z-bar 24a may be omitted and a thicker demister pad 24b may be used instead.

The air subsequently enters a UV disinfection chamber 26 having adequate UV energy for irradiating the flow of air. The treated air is released through the air outlet 30. The combined effect of the solution treatment and the UV treatment achieves a minimum 99.999% reduction of microorganisms. The UV irradiation in the present example is carried out by the use of an array of UV lamps 28a-c.

The liquid desiccant is propelled from the sump 16 to the distribution header 22 by a pump 32. The conditioning section 12 also incorporates liquid desiccant cooling via an externally mounted heat exchanger 34 located inbetween the pump 32 and the distribution header 22. The heat exchanger 34 controls temperature (±2°) and humidity (±1%) within the conditioned environment of the conditioning section 12. The humidity level is controlled via the temperature, as well as the concentration of the desiccant being sprayed from the spray nozzles 22a-e.

As the liquid desiccant absorbs water from the air, its ability to absorb more water, diminishes. Therefore, the system 10 also includes a regenerating section 12′ for regenerating the water absorbing properties of the desiccant solution. The regenerating section 12′ is similar to the conditioning section 12 and incorporates industrial-grade fiberglass construction with internal parts of non-metallic engineered plastics.

It should be noted that regeneration is not required to maintain the anti-bacterial properties of the solution. However, to maintain the dehumidifying properties of the solution, the solution must be pumped through a regenerator. In the present example, the solution is propelled from the sump 16 of the conditioning section 12 to the regenerating section 12′. In the present embodiment, a part of the flow which is propelled from the sump 16 to the distribution header 22 is redirected to the regenerating section 12′. A valve 36 is used for controlling the flow of solution from the conditioning section 12 to the regenerating section 12′. The regenerated liquid desiccant from the sump 16′ of the regenerating section 12′ is returned to the conditioning section 12 in a similar fashion controlled by a valve 36′. A heat exchanger 38 is used to equalise the temperature between the opposite flows of solution between the the conditioning section 12 and the regenerating section 12′.

In the regenerating section 12′, a separate flow of air is received from an air inlet 14′ and is drawn through a sump area 16′ with a 180° turn as shown by the arrow. A blower 40′ can be provided for generating the flow of air from the air inlet 14′ to an air outlet 30′. The sump area 16′ comprising the liquid desiccant. The flow then continues into a high efficiency contact packing chamber 18′ impregnated with liquid desiccant that allows for maximum surface contact of air to liquid desiccant. Downstream relative to the packing chamber 18′ and above the packing chamber 18′ when the regenerating section is in its normal position of use, a distribution chamber 20′ is located. The distribution chamber 20′ includes a solution spray distribution header 22′.The spray distribution header includes a drip tray or one or more spray nozzles 22a-e ′ for spraying liquid desiccant into the distribution chamber 20′ towards the packing chamber 18′.

Downstream relative to the distribution chamber 20 and above the distribution chamber 20 when the regenerating section 12′ is in its normal position of use, a droplet collection chamber 24′ is located. Solution is propelled from the sump 16′ to the distribution header 22′ by a pump 32′. The regenerating section 12′ also incorporates solution heating via a heat exchanger 34′ located inbetween the pump 32 and the distribution header 22. Heating the liquid desiccant will allow it to release water to the flow of air, which is released outdoor via an air outlet 30′.

The system 10 incorporates an electric and PLC (Programmable Logic Controller) control panel, fused disconnect, motor starters and level sensors. The PLC control panel incorporates a colour touch screen programmed with relay logic, diagnostic and loop control functions. A diagram of the dehumidification system is displayed on the screen indicating the status of the conditioning section fan, pump and outlet temperature; the regenerating section fan, pump, solution level and set points are also displayed. The conditioning section 12 incorporates temperature and pressure gauges with flanged connections. The regenerating section 12′ incorporates a control valve with electric operator for either steam or hot water; temperature and pressure gauges with flanged connections are also incorporated. Solution transfer modulating control valves with hand isolation are incorporated into both the conditioning and regenerating sections. A water makeup on/off control valve is also incorporated.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

We claim:

1. A system for neutralizing airborne microorganisms, comprising:
a conditioning section for accommodating a flow of air, the conditioning section comprising:
an air inlet for receiving microorganism laden air;
an air outlet for delivering microorganism free air;
a packing chamber located downstream relative to the air inlet, the packing chamber being filled with a fibrous packing material;
a distribution chamber located downstream relative to the packing chamber and located above the packing chamber when the system is in a normal position of use, the distribution chamber including at least one spray nozzle for spraying a liquid desiccant into the distribution chamber towards the packing chamber;
a sump located below the packing chamber when the system is in the normal position of use for receiving the liquid desiccant that has penetrated the packing chamber from the distribution chamber through gravity;
a pump for propelling the liquid desiccant from the sump to the at least one spray nozzle;
a droplet collection chamber located downstream relative to the at least one spray nozzle for removing droplets from the flow of air flowing through the droplet collection chamber; and
a UV disinfection chamber located downstream relative to the droplet collection chamber and upstream relative to the air outlet, the UV disinfection chamber comprising a UV energy source for exposing the flow of air to UV irradiation.

2. The system according to claim 1, wherein the liquid desiccant comprises a mixture of water and a salt, the salt being CaCl₂ or LiCl, and the concentration of the salt in the mixture being between 10% and 60%.

3. The system according to claim 2, wherein the concentration of the salt in the mixture is between 20% and 45%.

4. The system according to claim 1, wherein the UV energy source is an LED or a low-pressure UV lamp.

5. The system according to claim 4, wherein the low-pressure UV lamp is a low-pressure mercury lamp.

6. The system according to claim 1, wherein an intensity of UVC energy inside the UV disinfection chamber is at least 10 W/m².

7. The system according to claim 1, wherein an intensity of UVC energy inside the UV disinfection chamber is at least 50 W/m².

8. The system according to claim 1, wherein an intensity of UVC energy inside the UV disinfection chamber is at least 124 W/m².

9. The system according to claim 1, wherein the conditioner and/or internal parts are constructed of non-metallic plastics.

10. The system according to claim 1, wherein the droplet collection chamber includes a replaceable pad demister.

11. The system according to claim 1, further comprising a blower for generating the flow of air from the air inlet to the air outlet.

12. The system according to claim 11, wherein the flow of air defines a velocity less than 3 m/s.

13. The system according to claim 11, wherein the flow of air defines a velocity less than 2.235 m/s.

14. The system according to claim 1, wherein the system comprises a heat exchanger between the sump and the at least one spray nozzle for cooling the liquid desiccant.

15. The system according claim 1, wherein the exposure to UV irradiation occurs within a moving airstream downstream from the droplet collection chamber.

16. The system according to claim 1, wherein the UV disinfection chamber comprises UVC energy of 124 W/m² at air flow and a velocity less than 3 m/s for microbial inactivation.

17. The system according to claim 1, wherein the UV disinfection chamber comprise an array of UV energy sources.

18. The system according to claim 1, further comprising a regenerating section for regenerating the liquid desiccant.

19. The system according to claim 18, wherein the regenerating section extends between a secondary air inlet for dry air and a secondary air outlet for humid air and defines a secondary flow direction, the regenerating section comprising:
a secondary packing chamber located downstream relative to the air secondary inlet, the secondary packing chamber being filled with a secondary high efficiency packing material;
a secondary spray chamber located downstream relative to the secondary packing chamber and located above the secondary packing chamber when the system is in the normal position of use, the secondary spray chamber including at least one secondary spray nozzle for spraying the liquid desiccant into the secondary spray chamber towards the secondary packing section;
a secondary sump located below the secondary packing chamber when the system is in the normal position of use for receiving the liquid desiccant from the secondary spray chamber that has penetrated the secondary packing chamber through gravity;
a secondary pump for pumping the liquid desiccant from the secondary sump to the at least one secondary spray nozzle; and
a secondary heat exchanger between the secondary sump and the at least one secondary spray nozzle for heating the liquid desiccant.

20. A method of neutralizing airborne microorganisms comprising:
providing a conditioning section comprising:
an air inlet;
an air outlet;
a packing chamber located downstream relative to the air inlet, the packing chamber being filled with a fibrous packing material;
a distribution chamber located downstream relative to the packing chamber and located above the packing chamber when the system is in a normal position of use, the distribution chamber including at least one spray nozzle;
a sump located below the packing chamber when the system is in the normal position of use;
a pump;
a droplet collection chamber located downstream relative to the spray nozzle; and
an UV disinfection chamber located downstream relative to the droplet collection chamber and upstream relative to the air outlet, wherein the method comprises the steps of:
introducing microorganism laden air into the air inlet;
spraying a liquid desiccant into the distribution chamber towards the packing section by using an engineered spray system;
receiving the liquid desiccant that has penetrated the packing chamber from the distribution chamber through gravity into the sump, propelling the liquid desiccant from the sump to the at least one spray nozzle;
removing droplets from the air flowing through the droplet collection chamber;
exposing the air within the UV disinfection chamber to UV irradiation; and
receiving microorganism free air at the air outlet.
